(19) United States
(12) Patent Application Publication
(10) Pub. No.: US 2021/0093746 A1
(43) Pub. Date: Apr. 1, 2021

(54) AIR PURIFIER

(71) Applicant: Sunstar Engineering Inc., Osaka (JP)

(72) Inventors: Katsuhiro YAMAGUCHI, Osaka (JP); Yoshiki KIMURA, Osaka (JP)

(21) Appl. No.: 17/040,351

(22) PCT Filed: Jun. 25, 2020
(86) PCT No.: PCT/JP2020/024966

§ 371 Date: Sep. 22, 2020

(30) Foreign Application Priority Data
Sep. 26, 2019 (JP) 2019-175990

(51) Int. Cl. A61L 9/20; B01D 53/88

(52) U.S. Cl. CPC A61L 9/205; B01D 53/885; B01D 2255/802; B01D 2255/20707; B01D 2259/804

(57) ABSTRACT

Provided is an air purifier that allows efficient contact between air and a photocatalyst. An air purifier according to an aspect of the invention includes: a light source; a catalytic member which includes a mesh like carrier formed in a tubular shape and a photocatalyst supported on a surface of the carrier, and which is provided outside and spaced apart from the light source; a reflecting tube which is provided outside and spaced apart from the catalytic member, and which reflects light emitted from the light source and transmitted through the catalytic member; and a centrifugal blower which includes an impeller rotating substantially coaxially with respect to the reflecting tube and a housing having an intake port connected to one end of the reflecting tube.

### TECHNICAL FIELD

The present invention relates to an air purifier.

### BACKGROUND ART

An air purifier in practical use produces deodorizing and sanitizing effects by using a photocatalyst such as titanium oxide to decompose organic substances. Such an air purifier includes a catalytic member including a supported photocatalyst; a light source that emits light to the catalytic member to activate the photocatalyst; and an air flow generator that sucks air from around it and brings the air into contact with the photocatalyst.

In an air purifier using a photocatalyst, it is required that light is effectively applied to the photocatalyst to activate the photocatalyst sufficiently, and that air is efficiently brought into contact with the photocatalyst. For example, Patent Document 1 discloses “a photocatalytic air purifying apparatus including: a cylindrical container communicating with an air intake port and an air discharge port; a sirocco fan for sucking air from outside the container through the air intake port into the cylindrical container and for discharging the air from the air discharge port; an ultraviolet lamp that is provided on the center line of the air flow passage in the cylindrical container to apply ultraviolet rays toward the side surface of the container; and a carrier having a photocatalyst supported thereon, in which the carrier is disposed along the inner surface of the cylindrical container”.

The photocatalytic air purifying apparatus (air purifier) disclosed in Patent Document 1 is configured such that ultraviolet rays can be effectively applied from a rod-shaped ultraviolet lamp disposed on the center line of the cylindrical container to the photocatalyst supported on the carrier provided along the inner surface of the cylindrical container.

Patent Document 1: Japanese Unexamined Patent Application, Publication No. 2000-157838

### DISCLOSURE OF THE INVENTION

### Problems to be Solved by the Invention

The air purifier disclosed in Patent Document 1 has a sirocco fan with an outer diameter substantially equal to the inner diameter of a cylindrical container, which is provided at one inner end of the cylindrical container. The sirocco fan is a type of centrifugal blower, which includes an impeller having plural blades arranged cylindrically around the axis of rotation. The sirocco fan pushes out air from inside (rotation axis side) to outside the blades using the centrifugal force caused by the rotation of the impeller, and guides, in the circumferential direction, to discharge the air being pushed to outside the impeller, in the tangential direction. The sirocco fan sucks air from the vicinity of the axis of rotation at the center but cannot suck air from the outer periphery. Therefore, the air purifier disclosed in Patent Document 1 provides a relatively high air flow rate in the vicinity of the center of the cylindrical container and provides a relatively low air flow rate on the radially outer side, in other words, in the vicinity of the photocatalyst carrier. Accordingly, the air purifier disclosed in Patent Document 1 cannot provide a high contact efficiency between air and the photocatalyst, which will require an increase in ventilation frequency, in other words, an increased volume of air flow. Unfortunately, an increase in the volume of air flow may cause a problem such as large operating noise.

It is an object of the present invention to provide an air purifier that allows efficient contact between air and a photocatalyst.

### Means for Solving the Problems

An aspect of the present invention relates to an air purifier including: a light source; a catalytic member which includes a mesh like carrier formed in a tubular shape and a photocatalyst supported on a surface of the carrier, and which is provided outside and spaced apart from the light source; a reflecting tube which is provided outside and spaced apart from the catalytic member and which reflects light emitted from the light source and then transmitted through the catalytic member; and a centrifugal blower which includes an impeller rotating substantially coaxially with respect to the reflecting tube and a housing having an intake port connected to one end of the reflecting tube.

The air purifier according to an aspect of the present invention may further include an air discharge channel formation member which houses the centrifugal blower, which has an air discharge port opening substantially coaxial with the reflecting tube, and which defines an air discharge channel for guiding, to the air discharge port, air blown by the centrifugal blower; an air supply channel formation member which is disposed on a side of the reflecting tube opposite to the centrifugal blower; and an exterior member which is disposed outside and spaced apart from the reflecting tube and which has both ends sealed with the air discharge channel formation member and the air supply channel formation member.

In the air purifier according to an aspect of the present invention, the carrier may be multiply wound.

The air purifier according to an aspect of the present invention may further include a holder which is withdrawably inserted into the reflecting tube, and which holds the catalytic member.

In the air purifier according to an aspect of the present invention, the light source is held, at a centrifugal blower-side end of which, by a plurality of elastic members provided fixedly, and is fitted, at another opposite end of which, into the holder.

In the air purifier according to an aspect of the present invention, the holder may have a hooking portion that is disposed close to a centrifugal blower-facing side of the light source and that enables the light source to be withdrawn together with the holder from the reflecting tube.

### Effects of the Invention

The air purifier according to an aspect of the present invention allows air to efficiently come into contact with the photocatalyst.

### BRIEF DESCRIPTION OF THE DRAWINGS

FIG. 1 is a perspective view of an air purifier according to an embodiment of the present invention;

FIG. 2 is an exploded perspective view of the air purifier of FIG. 1;

FIG. 3 is an axial cross-sectional view of the air purifier of FIG. 1;

FIG. 4 is a cross-sectional view of the air purifier of FIG. 1 in a direction perpendicular to its axis;

FIG. 5 is an enlarged exploded perspective view of a reflecting tube for the air purifier of FIG. 1;

FIG. 6 is an enlarged perspective view of a holder for the air purifier of FIG. 1;

FIG. 7 is a graph showing the results of a test in which an air purifier is used to decompose acetaldehyde;

FIG. 8 is a graph showing the results of a test in which an air purifier is used to decompose acetic acid; and

FIG. 9 is a graph showing the results of a test in which an air purifier is used to decompose trimethylamine.

### PREFERRED MODE FOR CARRYING OUT THE INVENTION

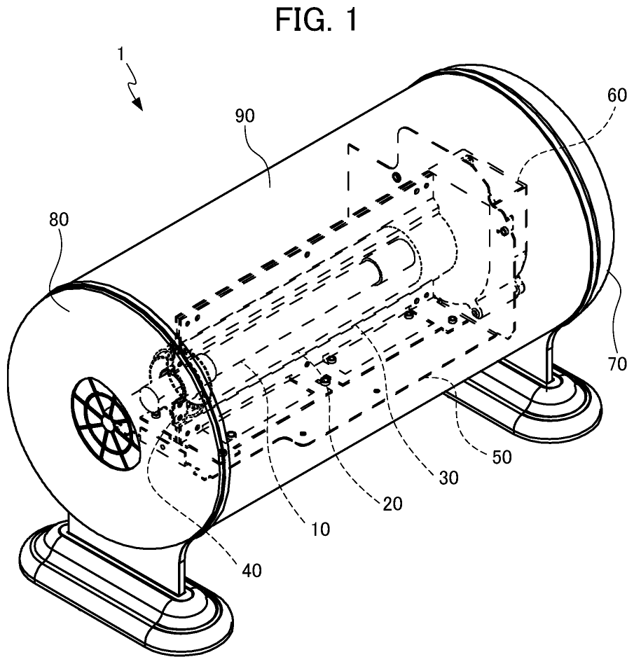

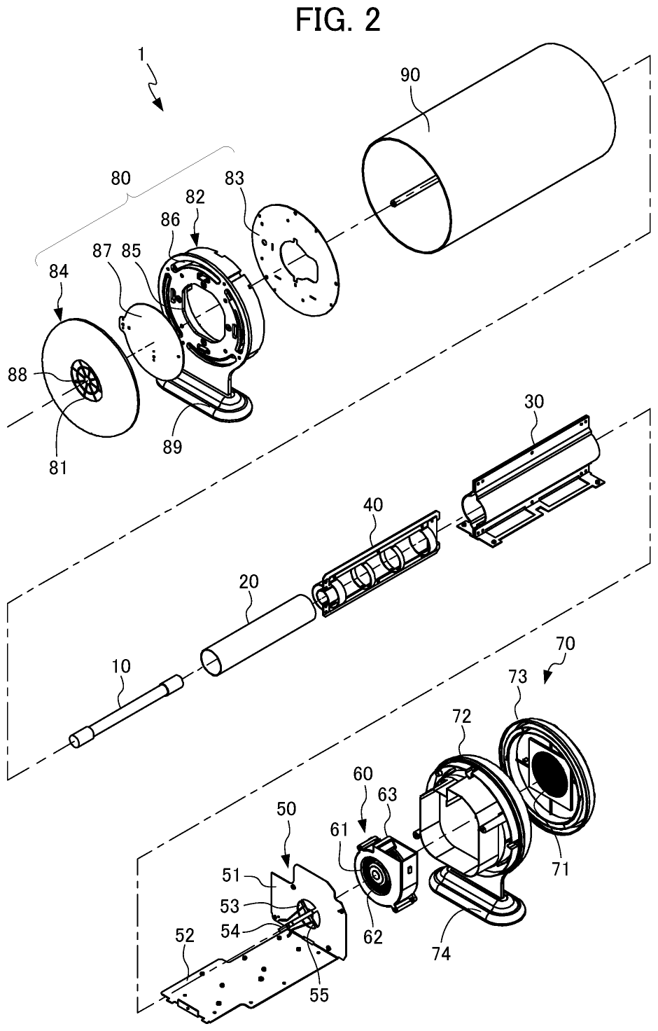

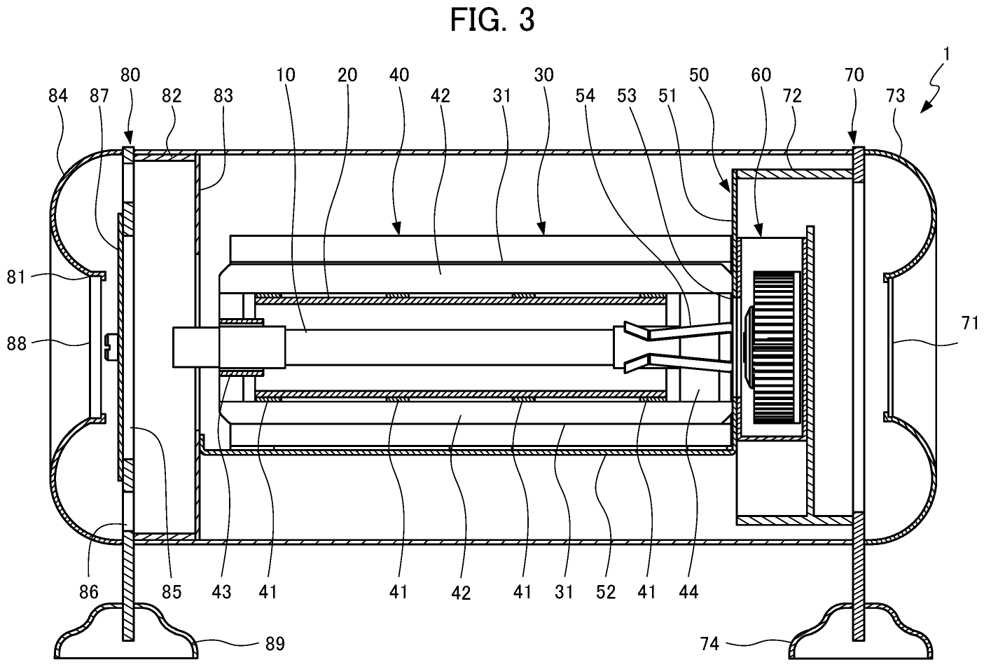

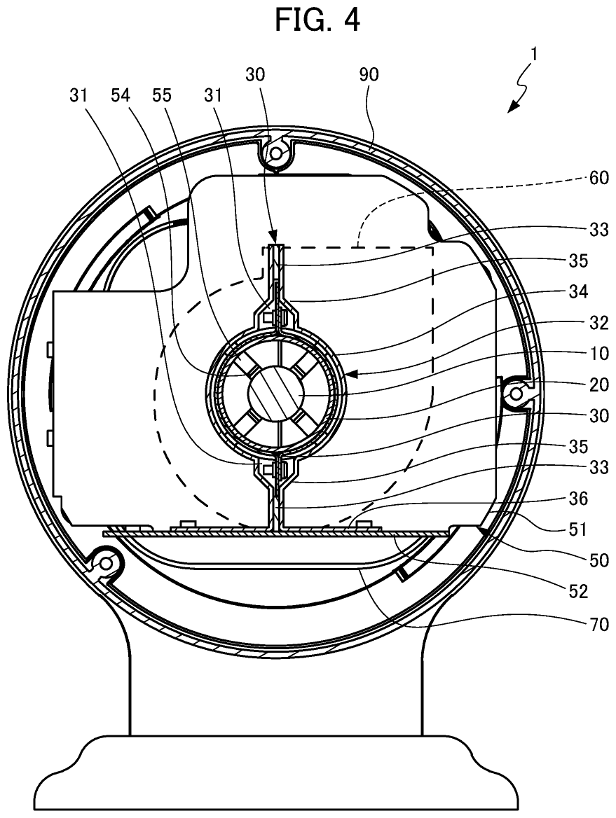

Hereinafter, embodiments of the present invention will be described with reference to the drawings. FIG. 1 is a perspective view of an air purifier 1 according to an embodiment of the present invention. FIG. 2 is an exploded perspective view of the air purifier 1. FIG. 3 is an axial cross-sectional view of the air purifier 1. FIG. 4 is a cross-sectional view of the air purifier 1 in a direction perpendicular to its axis. The air purifier 1 includes a light source 10, a catalytic member 20, a reflecting tube 30, a holder 40, a base member 50, a centrifugal blower 60, an air discharge channel formation member 70, an air supply channel formation member 80, and an exterior member 90.

The light source 10 is preferably in the form of a rod extending in a single direction (hereinafter referred to as the axial direction). The light source 10 preferably emits light substantially uniformly in all directions perpendicular to the axial direction. The light source 10 emits light, such as ultraviolet light, for activating a photocatalyst in the catalytic member 20, which will be described later. Specifically, the light source 10 is typically a rod-shaped ultraviolet lamp or a rod-shaped ultraviolet light emitting diode illuminator.

The catalytic member 20 includes a mesh like carrier formed in a tubular shape and a photocatalyst supported on the surface of the carrier. The catalytic member 20 as a whole is a mesh like tubular body having a large number of apertures through which light can pass. The catalytic member 20 is disposed outside and spaced apart from the light source, which will be described in detail later. As used herein, the term “mesh like” is intended to include any flat sheet having a large number of apertures, which is not limited to a member composed of warp and weft threads. An open ratio (the ratio of the area through which light can pass) of the catalytic member 20 may be 15% or more and 55% or less, and is preferably 20% or more and 50% or less. According to this feature, the light emitted from the light source 10 and passed through the catalytic member 20 can be reflected by the reflecting tube 30, described later, and then to be applied to the outer surface of the catalytic member 20, which is more effective in activating the photocatalyst of the catalytic member 20 and in decomposing organic substances and so on.

For instance, as the carrier, a metal sheet having fine apertures randomly formed by etching, a wire mesh, a molded resin mesh, a perforated metal sheet, and the like can be used, and in particular, an expanded metal is preferably used because it has high capacity to carry the photocatalyst and high mechanical properties and economical efficiency. If the carrier is made of titanium and the surface of the carrier is subjected to an oxidation treatment, an adhesion of the carrier and the photocatalyst can be improved. If the carrier is made of aluminum, the catalytic member 20 can be obtained with high workability at a relatively low cost.

The carrier is preferably multiply (double-layered in this embodiment) wound to form a tubular shape. By multiply winding the carrier with a high open ratio, catalytic member 20 can have a light transmittance in an appropriate range and a contact area between air and the photocatalyst can be increased. While the catalytic member 20 may be formed by depositing the photocatalyst on the surface of the carrier previously multiply wound, the catalytic member 20 may be formed by first depositing the photocatalyst on the surface of the carrier in the form of an elongated strip, then cutting the carrier into pieces with a suitable length, and winding each cut piece, which is a relatively easy process to obtain the catalytic member 20 in a desired shape. More specifically, by doubly winding a strip-shaped material having an open area ratio of 45% or more and 55% or less and including a carrier and a photocatalyst supported on the surface of the carrier, the catalytic member 20 can be formed with a suitable level of open ratio so as to allow efficient contact between air and the activated photocatalyst.

The photocatalyst may be, as a non-limiting example, anatase titanium oxide, which is currently known as a material with high photocatalytic activity. To have a large contact area with air, the photocatalyst is preferably in the form of particles or powder covering the surface of the carrier. The photocatalyst in that form may be fixed to the surface of the carrier by sintering. Specifically, by baking the carrier applied with slurry containing dispersed particles of the photocatalyst, a photocatalyst fixed to the surface of the carrier can be obtained.

A space between the light source 10 and the catalytic member 20 forms a main channel for air flow. As a lower limit of the distance between the light source 10 and the catalytic member 20, to ensure a sufficient air flow rate, 10 mm is preferable, and 15 mm is more preferable. On the other hand, as an upper limit of the distance between the light source 10 and the catalytic member 20, to achieve efficient contact between air and the photocatalyst, preferably has an upper limit of 50 mm is preferable, and 30 mm is more preferable.

As a lower limit of the thickness of the catalytic member 20, to ensure sufficient mechanical strength, 0.3 mm is preferable, and 0.5 mm is more preferable. On the other hand, as an upper limit of the catalytic member 20, to achieve efficient contact between the photocatalyst and the air flowing along the catalytic member 20, 3 mm is preferable, and 2 mm is more preferable.

As the axial length of the catalytic member 20 increases, while the contact between air and the photocatalyst increases so that organic substances and so on can be more effectively decomposed, but the volumetric efficiency decreases because the concentration of organic substances in the air decreases toward the downstream side. As a lower limit of the axial length of the catalytic member 20, to ensure sufficient contact between air and the photocatalyst, 5 times of the inner diameter of the catalytic member 20 are preferable, and 8 times of the inner diameter of the catalytic member 20 are more preferable. On the other hand, an upper limit of the axial length of the catalytic member 20, to make the air purifier 1 compact, 20 times are preferable, and 15 times are more preferable.

The reflecting tube 30 is formed in a substantially tubular shape, and disposed outside and spaced apart from the catalytic member 20. The reflecting tube 30 reflects the light emitted from the light source 10 and then transmitted through the catalytic member 20. This allows activation of the photocatalyst on the outer side of the catalytic member 20 and allows more effective decomposition of organic substances and so on.

The term “reflect light” is intended to mean to provide a reflectance of 50% or more at a wavelength that maximizes the absorbance by the photocatalyst. The inner surface of the reflecting tube 30 preferably has a light reflectance of 60% or more, more preferably 70% or more, at a wavelength that maximizes the absorbance by the photocatalyst. The higher the light reflectance of reflecting tube 30, the better for activation of the photocatalyst, however, it is not always preferred to have a mirror finish or the like on the reflecting tube 30 if the manufacturing cost and variations in performance due to pollution, etc. are considered. Specifically, the reflecting tube 30 may be formed by working a common metal material such as an aluminum sheet or a stainless steel sheet, and the reflecting tube 30 can be optimally designed on the premise of using such a material in order to obtain the air purifier 1 while the reduction in performance due to pollution, etc. is minimized. The reflection by the inner surface of the reflecting tube 30 may include not only specular reflection but also diffuse reflection.

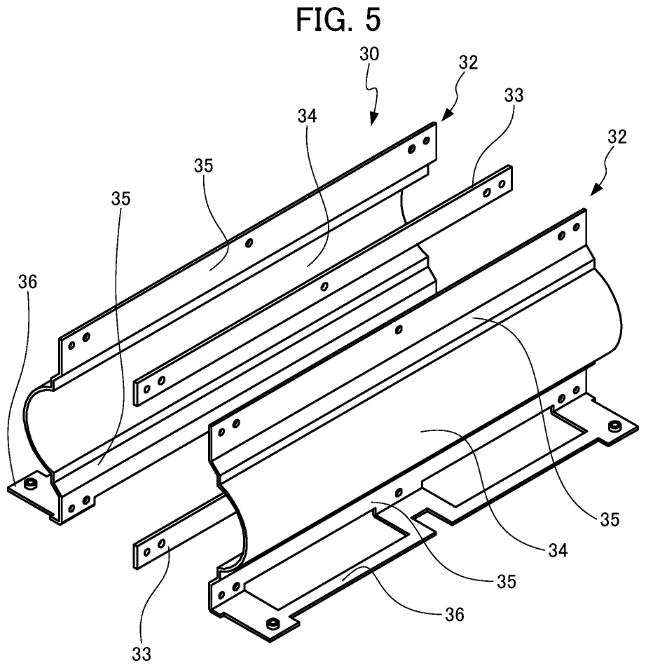

The reflecting tube 30 preferably has a receiving structure for receiving and supporting the holder 40, which will be described later. In an embodiment, the reflecting tube 30 has a pair of holding grooves 31, which are disposed in circumferentially symmetrical positions and each extend in the axial direction. As shown in FIG. 5 specifically, the reflecting tube 30 includes a pair of reflecting members 32 formed substantially symmetrical; and a pair of spacers 33 provided between the pair of reflecting members 32.

Each reflecting member 32 includes a cylindrical portion 34 which is formed by bending a metal sheet, has a half pipe-shaped reflecting surface for reflecting light emitted from the light source 10, a pair of holding groove-forming portions 35 which is angled and extends from the side edge of the cylindrical portion 34 to form a side wall of the holding groove 31, and an fixing portion 36 which extends from the holding groove-forming portion 35 and is attached to the base member 50.

As shown in the drawing, the holding groove-forming portion 35 may be bent to conform to the shape of the holder 40. The holding groove-forming portion 35 of the present embodiment is bent to form a step portion along the extending direction so that the step portion increases the width of the opening of the holding groove 31 to accommodate the screws used to assemble the parts of the holder 40. The spacer 33 is a plate-shaped member that defines the width of the top or bottom portion of the holding groove 31.

As a lower limit of the distance between the catalytic member 20 and the reflecting tube 30, equal to, more preferably, equal to the thickness of the catalytic member 20 is preferable, and 1.5 times of the thickness of the catalytic member 20 is more preferable, so that non-aperture portions of the catalytic member 20 can be irradiated with the light transmitted through the apertures of the catalytic member 20 and then reflected from the reflecting tube 30. On the other hand, As an upper limit of the distance between the catalytic member 20 and the reflecting tube 30, 10 mm is preferable, and 5 mm is more preferable so that the catalytic member 20 can have a relatively large area (diameter). In other words, if the distance between the catalytic member 20 and the reflecting tube 30 is too short, the light may fail to reach the photocatalyst on the outer side of the catalytic member 20 since the specularly reflected light may pass back through the apertures of the catalytic member 20. On the contrary, if the distance between the catalytic member 20 and the reflecting tube 30 is too long, the catalytic member 20 should have a small diameter relative to the inner diameter of the reflecting tube 30, which is determined according to the type of the centrifugal blower 60 described later, so that the catalytic member 20 has a relatively small effective area.

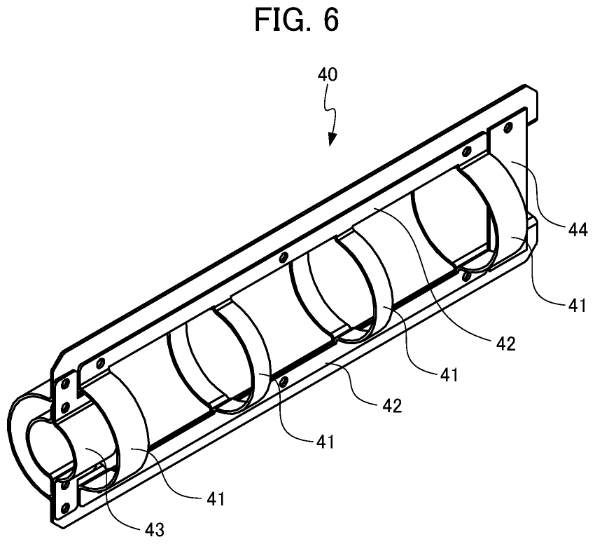

The holder 40 holds the catalytic member 20, and is withdrawablely inserted from, in the axial direction, the reflecting tube 30. As shown in FIG. 6 specifically, the holder 40 can be configured to include plural band portions 41 which are arranged to wrap the circumference of the catalytic member 20, and a pair of joint portions 42 which extend in the axial direction to join the plural band portions 41 and which fit in the holding groove 31 of the reflecting tube 30. These features make it possible to remove and clean the catalytic member 20 easily.

The holder 40 may include an annular holding portion 43 which holds one end of the light source 10 opposite to the centrifugal blower 60, and a hooking portion 44 which is provided close to the centrifugal blower 60-facing side of the light source 10 and enable to withdraw the light source 10 together with the holder 40 from the reflecting tube 30. These features make it possible to easily remove the light source 10 for replacement or cleaning. In addition, the centrifugal blower 60-facing end of the light source 10 may be held by the base member 50, which will be described below.

The base member 50 fixes the reflecting tube 30, the centrifugal blower 60, the air discharge channel formation member 70, and the air supply channel formation member 80. Specifically, the base member 50 may be configured to include a plate-shaped vertical portion 51 provided between the reflecting tube 30 and the centrifugal blower 60, and an extending portion 52 which extends from the vertical portion 51 in the axial direction and to which the reflecting tube 30 is attached.

The vertical portion 51 connects the centrifugal blower 60 to the reflecting tube 30 serving as an air flow channel. For this purpose, the vertical portion 51 has an opening 53 for allowing air to pass therethrough. Thus, the centrifugal blower 60 only sucks the air that has passed through the inside of the reflecting tube 30. The inner edge of the opening 53 is shaped to substantially match the end face of the cylindrical portion 34 of the reflecting tube 30.

The vertical portion 51 may be provided with plural elastic members 54, which are attached thereto to hold the centrifugal blower 60-facing end of the light source 10. For this purpose, the vertical portion 51 may have crossing portions 55, for example, in the form of a strip, which cross the opening 53 and to which the elastic members 54 are attached. In other words, the opening 53 may be divided into plural sections with the crossing portions 55 as the remainder. The plural elastic members 54 are disposed so as not to interfere with the hooking portion 44 of the holder 40.

The elastic members 54 are formed to extend opposite to the centrifugal blower 60 and to get close to each other by their elastic forces, and respectively have a distal end portion bent outward such that the distance between their distal ends is larger than the outer diameter of the end of the light source 10. Thus, one end of the light source 10 can be held between the elastic members 54 by inserting the light source 10 into the space between the elastic members 54 in the axial direction. Specifically, the light source 10 can be attached to a suitable place in the air purifier 1, by inserting the light source 10, in a state which the holder 40 holding the catalytic member 20 is previously held inside of the reflecting tube 30, into the holding portion 43 of the holder 40, and then pushing the light source 10 until one end is held between the elastic members 54 and the other end is fitted in the holding portion 43.

The centrifugal blower 60 includes an impeller 61 rotated substantially coaxially with respect to the reflecting tube 30 and a housing 63 formed with an intake port 62 connected to one end of the reflecting tube 30. The centrifugal blower 60 sucks air in the axial direction, moves the air radially outward trough the impeller 61, and discharges the air in the tangential direction of the impeller 61. Specifically, the centrifugal blower 60 may be a plate fan (radial blower or radially bladed fan), a sirocco fan (multi-blade blower fan or forward curved bladed fan), or a turbofan (backward curved bladed fan). In particular, a sirocco fan is preferably used. In this regard, the expression “substantially coaxially” means that the rotational center line of the impeller 61 only has to pass through the interior space of the reflecting tube 30 with no need for an exact match between the axes.

By connecting one end of the reflecting tube 30 to the intake port 62 of the centrifugal blower 60 with the vertical portion 51 of the base member 50 placed between them, it is possible to use a commercially available product as the centrifugal blower 60, which does not have any structure for connecting the flow channel to the intake port 62, and thus the cost of the air purifier 1 can be lowered.

The centrifugal blower 60 is substantially directly connected to the intake port 62, and sucks air through the reflecting tube 30 extending in the axial direction, which serves as a suction flow channel. Therefore, the air linearly flows from the reflecting tube 30 into the centrifugal blower 60, so that the air flow forms along the catalytic member 20 inside the reflecting tube 30 to facilitate the contact between the air and the photocatalyst.

More specifically, by means of the air flow along the catalytic member 20, micro flows which enter the mesh of the catalytic member 20 can occur due to the irregularities (mesh geometry) of the catalytic member 20 and the viscosity of the air. Thus, by means of the air flow along the catalytic member 20, the air comes into contact with the photocatalyst present inside the apertures of the catalytic member 20, so that the power necessary for effective contact between the air and the photocatalyst can be lower than that necessary when the air flow is generated so as to pass through the catalytic member 20. In addition, the centrifugal blower 60 can have ample performance, since the intake port 62 has a diameter substantially equal to that of the reflecting tube 30 and the impeller 61 of the centrifugal blower 60 has an outer diameter larger than the inner diameter of the reflecting tube 30. Therefore, it is possible to reduce the operating noise from the centrifugal blower 60.

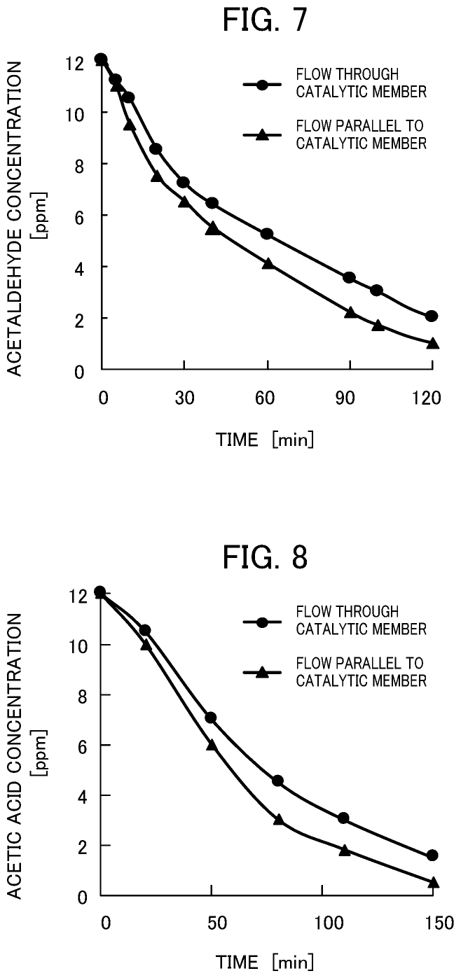

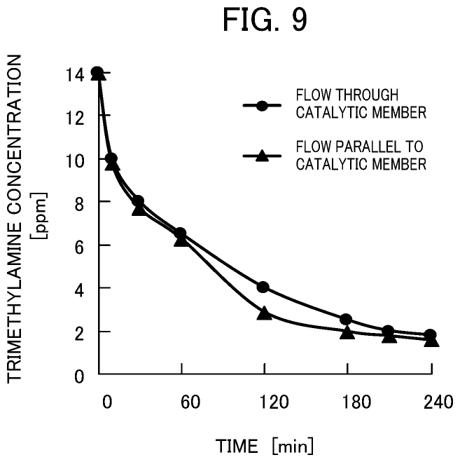

FIGS. 7 to 9 show data from experiments comparing changes in the concentration of acetaldehyde, acetic acid, and trimethylamine when the air is allowed to pass through the catalytic member 20 and when the air is allowed to flow along the catalytic member 20. Experimental conditions other than the air flow route, such as the intensity of light (including the reflected light) and the volume of air flow, were adjusted to remain the same.

The results have demonstrated that the air flow along the catalytic member 20 allows more efficient decomposition of a variety of odor components.

The air discharge channel formation member 70 houses the centrifugal blower 60, and has an air discharge port 71 opening substantially coaxial with the reflecting tube 30 on a side of the centrifugal blower 60 opposite to the reflecting tube 30. The air discharge channel formation member 70 defines an air discharge channel for guiding the air discharged by the centrifugal blower 60 to the air discharge port 71. The air discharge channel formation member 70 preferably forms a flow channel that force the air discharged from the centrifugal blower 60 to collide with the inner wall so that the dynamic pressure turns into a static pressure. This allows substantially uniform discharge of the air from the whole of the air discharge port 71. Furthermore, the centrifugal blower 60 can be disposed so as not to be directly viewed from the air discharge port 71, which reduces the transfer of the operating noise from the centrifugal blower 60 to the outside.

Specifically, the air discharge channel formation member 70 can be configured to have, an inner portion 72 that house the centrifugal blower 60 and that convert the dynamic pressure of the air discharged from the centrifugal blower 60 into a static pressure, and an outer portion 73 that guides the air flown out from the inner portion 72 to the air discharge port 71.

The air supply channel formation member 80 is disposed on a side of the reflecting tube 30 opposite to the centrifugal blower 60. The air supply channel formation member 80 has an air supply port 81 having an opening substantially coaxial with the reflecting tube 30. The air supply channel formation member 80 may include an Inner portion 82, a sealing plate 83, and an outer portion 84. The Inner portion 82 is attached to the base member 50 so as to be adjacent to the reflecting tube 30. The sealing plate 83 is provided to seal an outer peripheral portion of the Inner portion 82 as viewed in the axial direction of the reflecting tube 30. The outer portion 84 is connected to a side of the Inner portion 82 opposite to the reflecting tube 30. The outer portion 84 has an air supply port 81.

The Inner portion 82 preferably has an attachment/detachment opening 85 to attach and detach the holder 40 as well as light source 10 and the catalytic member 20 held by the holder 40 in a central portion, and flow channel openings 86 to allow the air sucked through the air supply port 81 to pass through in outer peripheral portion, in axial view. The Inner portion 82 forms a flow channel that guides the air passed through the flow channel openings 86 to the reflecting tube 30.

The air supply channel formation member 80 further has a light source cover 87 that closes the attachment/detachment opening 85 to blocks the air passed through the flow channel openings 86 during the operation of the air purifier 1. Preferably, the light source cover 87 is detachably attached to the inner portion 82 with knobbed screws, thumb screws, or other means. If an interlocking mechanism may be further provided to prevent the light source 10 from emitting light unless the light source cover 87 is attached, the user is prevented from accidentally directly viewing the light emitted from the light source 10.

The outer portion 84 defines a flow channel that guides the air passed through the air supply port 81 to the flow channel openings 86. Furthermore, the air supply channel formation member 80 preferably has a filter 88 provided at the flow channel openings 86.

The air purifier 1 according to an embodiment has a pair of leg members 74 and 89 extending from the air discharge channel formation member 70 and the air supply channel formation member 80, respectively. The leg members 74 and 89 can be used to mount the air purifier 1 on a floor surface and can also be used as brackets for attaching the air purifier 1 to a wall surface.

The exterior member 90 is disposed outside and spaced apart from the reflecting tube 30, and is in a tubular shape having both ends sealed with the air discharge channel formation member 70 and the air supply channel formation member 80. The space between the reflecting tube 30 and the exterior member 90 contains a controller and a power circuit (not shown) for supplying power to the light source 10 and the centrifugal blower 60. Specifically, with the air purifier 1 being designed such that, in axial view, the sizes of the air supply channel formation member 80 and the exterior member 90 conform with the size of the air discharge channel formation member 70 necessary to use the centrifugal blower 60 and such that electric parts are placed in the space formed based on the difference between the sizes of the air discharge channel formation member 70 and the intake port 62 of the air centrifugal blower 60 as well as the reflecting tube 30, a design of the air purifier 1 can be improved.

As described above, the air purifier 1 according to an embodiment allows air to flow along the catalytic member 20 through the reflecting tube 30 with one end connected to the intake port 62 of the centrifugal blower 60, and therefore, the air is efficiently brought into contact with the photocatalyst for decomposing organic substances. In the air purifier 1, since the air flow along the catalytic member 20, the resistance of air flow pass is low, resulting in low operating noise from the centrifugal blower 60 and low power consumption of the centrifugal blower 60.

While the air purifier 1 according to an embodiment of the present invention has been described, it will be understood that the features and advantageous effects of the air purifier according to the present invention are not limited to those described above. For example, in the air purifier according to the present invention, the air supply port and the air discharge port may be provided non-coaxially with the light source and may be provided to supply and discharge the air in the radial direction.

### EXPLANATION OF REFERENCE NUMERALS

1: Air purifier
10: Light source
20: Catalytic member
30: Reflecting tube
31: Holding groove
32: Reflecting member
33: Spacer
34: Cylindrical portion
35: Groove-forming portion
36: Fixing portion
40: Holder
41: Band portion
42: Joint portion
43: Holding portion
44: Hooking portion
50: Base member
51: Vertical portion
52: Extending portion
53: Opening
54: Elastic member
55: Crossing portion
60: Centrifugal blower
61: Impeller
62: Intake port
63: Housing
70: Air discharge channel formation member
71: Air discharge port
72: Inner portion
73: Outer portion
74: Leg member
80: Air supply channel formation member
81: Air supply port
82: Inner portion
83: Sealing plate
84: Outer portion
85: Attachment/detachment opening
86: Flow channel opening
87: Light source cover
88: Filter
89: Leg member
90: Exterior member

1. An air purifier comprising:
a light source;
a catalytic member which includes a mesh like carrier formed in a tubular shape and a photocatalyst supported on a surface of the carrier, and which is provided outside and spaced apart from the light source;
a reflecting tube which is provided outside and spaced apart from the catalytic member, and which reflects light emitted from the light source and then transmitted through the catalytic member; and
a centrifugal blower which includes an impeller rotating substantially coaxially with respect to the reflecting tube and a housing having an intake port connected to one end of the reflecting tube.

2. The air purifier according to claim 1, further comprising:
an air discharge channel formation member which houses the centrifugal blower, which has an air discharge port opening substantially coaxial with the reflecting tube, and which defines an air discharge channel for guiding, to the air discharge port, air blown by the centrifugal blower;
an air supply channel formation member which is disposed on a side of the reflecting tube opposite to the centrifugal blower and which has an air supply port opening substantially coaxial with the reflecting tube; and
an exterior member which is disposed outside and spaced apart from the reflecting tube, and which has both ends sealed with the air discharge channel formation member and the air supply channel formation member.

3. The air purifier according to claim 1, wherein, the catalytic member is formed in a manner that multiply winds the carrier.

4. The air purifier according to claim 1, further comprising a holder which is withdrawably inserted into the reflecting tube, and which holds the catalytic member.

5. The air purifier according to claim 4, the light source is held, at a centrifugal blower-side end of which, by a plurality of elastic members provided fixedly, and is fitted, at another opposite end of which, into the holder.

6. The air purifier according to claim 5, wherein the holder has a hooking portion that is disposed close to a centrifugal blower-facing side of the light source, and that enables the light source to be withdrawn together with the holder from the reflecting tube.
